(19) United States
(12) Patent Application Publication
(10) Pub. No.: US 2017/0128252 A1
(43) Pub. Date: May 11, 2017

(54) FRAGMENT SPECIFIC FRACTURE REDUCTION BRACE AND ASSOCIATED METHOD OF USE

(71) Applicant: Barnaby Todd DEDMOND, Chapin, SC (US)

(72) Inventor: Barnaby Todd DEDMOND, Chapin, SC (US)

(21) Appl. No.: 15/343,567

(22) Filed: Nov. 4, 2016

Related U.S. Application Data
(60) Provisional application No. 62/251,172, filed on Nov. 5, 2015.
(60) Provisional application No. 62/371,975, filed on Aug. 8, 2016.

(51) Int. Cl. A61F 5/058

(52) U.S. Cl. CPC A61F 5/05875; A61F 5/05866

(57) ABSTRACT

The present invention provides a method for providing and using a fracture reduction brace, comprising: providing one or more rigid splint members configured to be disposed adjacent to or about an anatomical structure; providing one or more reduction members deployable from an interior surface of the one or more rigid splint members into the anatomical structure such that the one or more reduction members apply pressure to the anatomical structure thereby reducing an associated fracture; disposing the one or more rigid split members securely adjacent to or about the anatomical structure; and deploying the one or more reduction members from the interior surface of the one or more rigid splint members into the anatomical structure.

### CROSS-REFERENCE TO RELATED APPLICATIONS

The present patent application/patent claims the benefit of priority of co-pending U.S. Provisional Patent Application No. 62/251,172, filed on Nov. 5, 2015, and entitled “BOXER'S FRACTURE REDUCTION BRACE,” and U.S. Provisional Patent Application No. 62/371,975, filed on Aug. 8, 2016, and entitled “FRAGMENT SPECIFIC FRACTURE REDUCTION BRACE,” the contents of both of which are incorporated in full by reference herein.

### FIELD OF THE INVENTION

The present invention relates generally to braces for use in the immobilization of a fracture. More specifically, the present invention relates to fragment specific braces for use in the reduction and immobilization of a fracture.

### BACKGROUND OF THE INVENTION

The concept of using a brace to treat a fracture is not new. Nondisplaced fractures of the hand, wrist, forearm, elbow, humerus, shoulder, foot, ankle, tibia, and knee are all at times treated with braces. In most situations, however, the brace is designed simply to hold the fracture in place, and not to reduce the fracture. There are certain situations, such as with the humeral shaft and, less commonly, the tibia, in which a brace is actually used to reduce a fracture. However, in these instances, the braces only provide coarse adjustment by circumferentially squeezing the soft tissue envelope around the fracture in order to improve reduction. Even in these instances, this is inadequate.

By way of specific example, fractures of the 5^{th} metacarpal neck are common fractures seen every day in orthopaedic offices. Frequently the result of punching a stationary object or another person, the so-called “boxer's fracture” has a typical fracture pattern with apex dorsal angulation. These fractures are treated in one of three ways: 1) casting in situ (leaving the fracture angulated and placement in a cast extending from the mid forearm to the tips of the 4^{th} and 5^{th} fingers), 2) closed reduction and casting (pushing the fracture back into appropriate alignment and then placement in a cast extending from the mid forearm to the tips of the 4^{th} and 5^{th} fingers); or 3) closed reduction with percutaneous pinning and casting (pushing the fracture back into appropriate alignment, surgically placing a pin across the fracture to stabilize it, and then placement in a cast extending from the mid forearm to the tips of the 4^{th} and 5^{th} fingers). Whenever a closed reduction is performed in the office or in the operating room, it is done in two stages. First, the fracture is reduced. Then, the reduction has to be let go of while a cast is placed. The cast is molded, essentially performing a second reduction. After the cast hardens, an x-ray is taken to determine if the fracture is in appropriate alignment. If the alignment is not good, the surgeon and patient then decide whether to repeat the process hoping for a better outcome, accept the alignment as is, or to proceed with surgical stabilization with a pin. Again, this is a cumbersome and inadequate process, taking too much time, resulting in too much pain, and yielding marginal results, at best.

Thus, what are still needed in the art are improved fragment specific braces for use in the reduction and immobilization of a fracture.

### BRIEF SUMMARY OF THE INVENTION

In various exemplary embodiments, the present invention provides a fracture reduction bracing system that not only allows for the coarse adjustment provided by currently available braces, but also provides for the fragment specific reduction of fractures unlike anything else available. This fragment specific fracture reduction bracing system is based around a series of adjustable fracture reduction pads that can be slid into a specific location for a given fracture and deployed in an appropriate amount to provide the specific forces in the specific direction with the specific amount of force that is required for reducing the fracture and maintaining that reduction until the fracture has healed. While this technology can be utilized for a wide variety of fracture reductions, specific braces under development utilizing this fragment specific fracture reduction technology include braces for the 4^{th} and 5^{th} metacarpals, distal radius, forearm, supracondylar humerus, humeral shaft, proximal humerus, proximal tibia, tibial shaft, and ankle. Presented are representative designs for the 4^{th} and 5^{th} metacarpals (boxer's brace), distal radius/forearm (pediatric forearm fracture reduction brace), and humeral shaft (humeral fracture reduction brace).

The boxer's fracture reduction brace (BFRB), for example, provides a significant improvement over existing methods of treatment of this fracture, as described herein. The BFRB allows for a single step reduction. The brace is placed, and using a simple screw in mechanism, the fracture is reduced. The brace remains in place as the final stabilizing device. No cast is necessary. Like casting, x-rays are taken at this time to assess reduction. If not adequate, simply providing a couple of more turns of the screw in mechanism provides greater reduction force and recheck is performed using the x-ray. It is very simple, quick, and easy and allows a doctor to dial in a perfect reduction every time. Also, with the enhanced stability, the brace can be a hand based brace, allowing significantly improved function as compared to a forearm based cast. The brace can be made of materials that can get wet, allowing showering, an improvement over most casts.

For the doctor, this dramatically simplifies the reduction process, saving time. For the patient, this provides the ability to dial in a perfect alignment every time, which improves patient outcomes. A hand based splint allows much greater function than a forearm based cast, while still providing adequate stability to the fracture. Again, the materials of the brace allow for showering, etc.

In one exemplary embodiment, the present invention provides a fracture reduction brace, comprising: one or more rigid splint members configured to be disposed adjacent to or about an anatomical structure; and one or more reduction members deployable from an interior surface of the one or more rigid splint members into the anatomical structure such that the one or more reduction members apply pressure to the anatomical structure thereby reducing an associated fracture. Optionally, the one or more rigid splint members comprise a plurality of rigid splint members. Optionally, the plurality of rigid split members are coupled together via one or more straps or other coarse adjustment mechanisms. Optionally, the plurality of rigid split members are coupled together via one or more structural members. Each of the one or more reduction members is deployable from the interior surface of an associated rigid splint member via an actuation mechanism that protrudes through the associated rigid splint member. Optionally, the actuation mechanism comprises a screw assembly that is actuated via rotational motion. Each of the one or more reduction members is translatable along the interior surface of an associated rigid splint member. Optionally, each of the one or more reduction members is translatable along the interior surface of the associated rigid splint member via a groove manufactured into the associated rigid splint member. The brace also comprises one or more compliant pads coupled to the interior surface of the one or more rigid splint members. The brace further comprises one or more compliant pads coupled to the one or more reduction members.

In another exemplary embodiment, the present invention provides a method for providing and using a fracture reduction brace, comprising: providing one or more rigid splint members configured to be disposed adjacent to or about an anatomical structure; providing one or more reduction members deployable from an interior surface of the one or more rigid splint members into the anatomical structure such that the one or more reduction members apply pressure to the anatomical structure thereby reducing an associated fracture; disposing the one or more rigid split members securely adjacent to or about the anatomical structure; and deploying the one or more reduction members from the interior surface of the one or more rigid splint members into the anatomical structure. Optionally, the one or more rigid splint members comprise a plurality of rigid splint members. Optionally, the plurality of rigid split members are coupled together via one or more straps or other coarse adjustment mechanisms. Optionally, the plurality of rigid split members are coupled together via one or more structural members. Each of the one or more reduction members is deployable from the interior surface of an associated rigid splint member via an actuation mechanism that protrudes through the associated rigid splint member. Optionally, the actuation mechanism comprises a screw assembly that is actuated via rotational motion. Each of the one or more reduction members is translatable along the interior surface of an associated rigid splint member. Optionally, each of the one or more reduction members is translatable along the interior surface of the associated rigid splint member via a groove manufactured into the associated rigid splint member. The brace also comprises one or more compliant pads coupled to the interior surface of the one or more rigid splint members. The brace further comprises one or more compliant pads coupled to the one or more reduction members.

### BRIEF DESCRIPTION OF THE DRAWINGS

The present invention is illustrated and described herein with reference to the various figures, in which like reference numbers are used to denote like assembly components/method steps, as appropriate, and in which:

FIG. 1 is a schematic diagram conceptually illustrating the fragment specific fracture reduction brace of the present invention (not specific to any given type of fracture);

FIG. 2 is a schematic diagram conceptually illustrating one exemplary embodiment of the BFRB of the present invention;

FIG. 3 is a photograph illustrating a prototype of the BFRB of the present invention;

FIG. 4 is another photograph illustrating a prototype of the BFRB of the present invention;

FIG. 5 is a further photograph illustrating a prototype of the BFRB of the present invention;

FIG. 6 is a schematic diagram conceptually illustrating one exemplary embodiment of the pediatric forearm fracture reduction brace (PFFRB) of the present invention;

FIG. 7 is a perspective diagram (vertical view) illustrating one exemplary embodiment of the humeral fracture reduction brace (HFRB) of the present invention;

FIG. 8 is another perspective diagram (end view) illustrating one exemplary embodiment of the HFRB of the present invention (inner piece); and

FIG. 9 is a further perspective diagram (end view) illustrating one exemplary embodiment of the HFRB of the present invention (lateral piece).

### DETAILED DESCRIPTION OF THE INVENTION

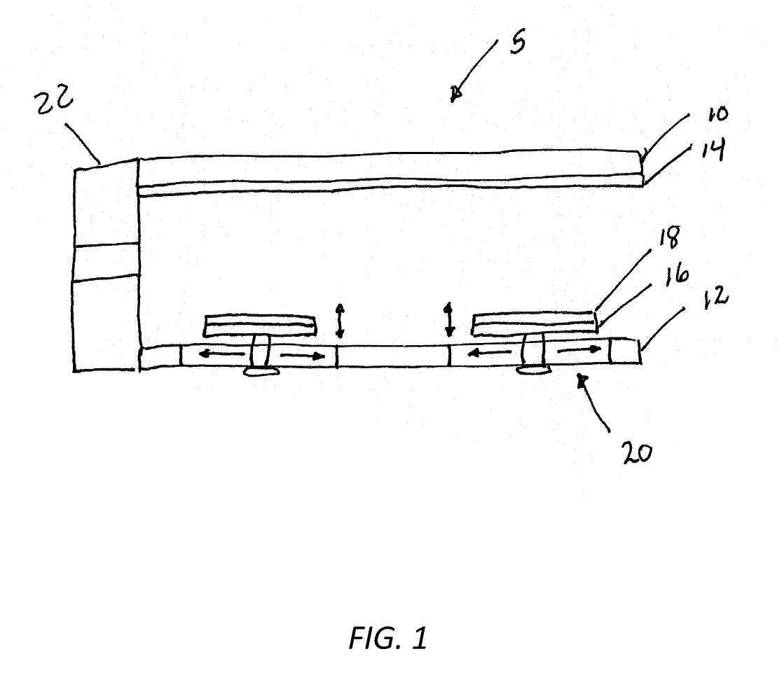

Referring now specifically to FIG. 1, in a broad conceptual embodiment, the fragment specific reduction brace 5 of the present invention includes a plurality of splint members 10 and 12 that are shaped and sized to accommodate a given anatomical structure. Preferably, each of the splint members 10 and 12 are made from a durable rigid material that can get wet, and one or more of the splint members 10 and 12 may include one or more compliant pads 14 that contact the anatomical structure for comfort. One or more reduction members 16 are deployable from the interior surface of one or more of the splint members 10 and 12, such that the one or more reduction members 16 apply selective pressure on and provide positioning of one or more portions of the anatomical structure. This adjustably manipulates and reduces the anatomical structure consistently while the brace 5 is in place, as verified by one or more x-rays or the like. Again, each of the one or more reduction members 16 may include one or more compliant pads 18 that contact the anatomical structure for comfort. The reduction members 16 may be translated along the associated splint member 10 or 12 via movement within a slot 20 manufactured into the splint member 10 or 12, for example. The reduction members 16 may be deployed by rotational actuation of a screw or jack mechanism, for example, and prevented from translating via a suitable locking mechanism or the like. One or more localized air pockets may also be utilized, provided that an associated reduction member 16 is thereby deployed and positioned. Finally, the one or more splint members 10 and 12 may be coupled together via one or more rigid structural members or compliant strap members 22, any of which may be adjustable. In this respect, the one or more splint members 10 and 12 may be formed together, defining one or more voids.

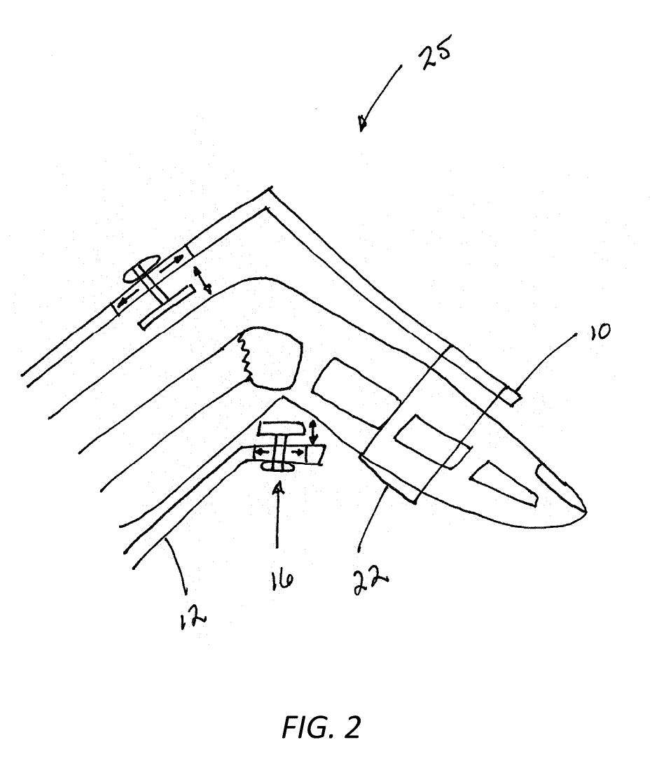

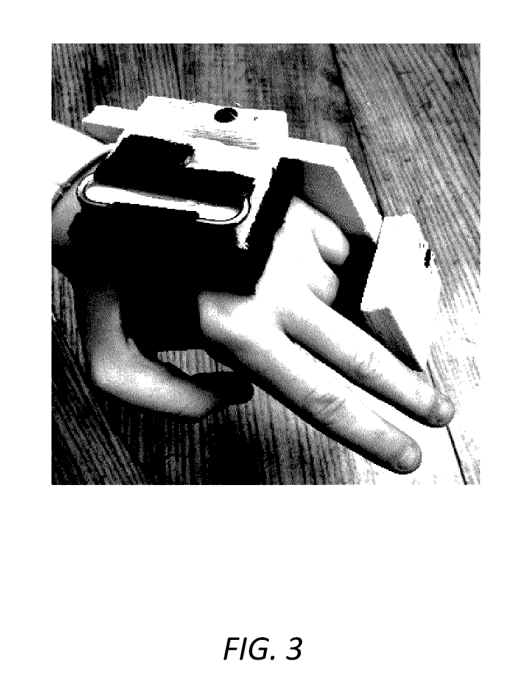

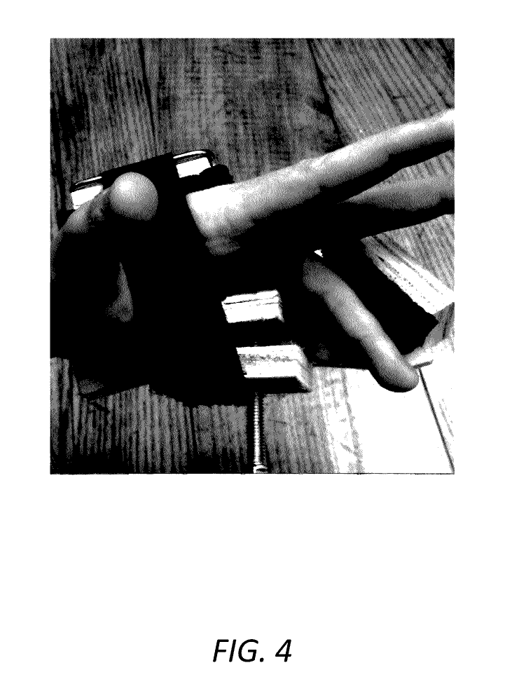

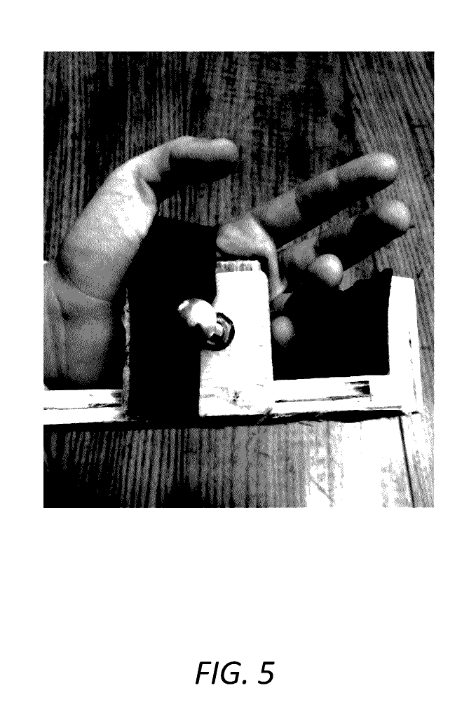

Referring now specifically to FIG. 2, in one exemplary embodiment, the BFRB 25 of the present invention includes a rigid dorsal plate 10 and a volar plate 12 that are slid over the ulnar border of the hand with a bar holding the fingers at approximately 70 degrees (a comfortable intrinsic plus position). Accordingly, the dorsal plate 10 and the volar plate 12 may each include multiple members disposed at angles to one another, such as a 70 degree angle, a 45 degree angle, etc. An adjustable plate 16 on the volar side is screwed into place, which applies pressure to the volar aspect of the metacarpal head, pushing it into a reduced position. Once appropriately reduced, screwing is stopped and thus the fracture is held in a reduced position. Other adjustable plates 16 may be provided as appropriate. An adjustable strap or the like 22 through the 1^{st} web space holds device 25 securely on the hand. Other adjustable straps or the like 22 may be provided as appropriate. A prototype of the BFRB 25 is illustrated in FIGS. 3-5.

There are approximately 160,000 metacarpal fractures per year in the United States. Approximately one-half of these are likely boxer's fractures. These could be treated with the BFRB 25 if it were available.

The PFFRB of the present invention simultaneously reduces and immobilizes fractures of the pediatric forearm from the midshaft of the forearm to the distal radius. It is more reproducible, easier and quicker to apply, and potentially better tolerated as compared to traditional casting, while obtaining and maintaining a better reduction. It provides better comfort for the patient as it is fully waterproof, allowing swimming and bathing. It also provides enhanced stability of the fracture reduction pads, allowing the elbow to be left free, making the brace more comfortable than long arm casting. It further provides better alignment for obtaining reduction, including precise adjustment of the fracture reduction pads, allowing for more precise application of reduction pressure as compared to traditional molding of a cast, thus often improving the initial reduction. It still further provides easy fine tuning of the pressure applied by the fracture reduction pads, allowing for easy correction when the fracture is not quite perfectly reduced. The surgeon can literally dial in the reduction under fluoroscopic guidance. Better maintenance of the reduction is provided—as swelling subsides, fine tuning of the fracture reduction pad is simple, allowing for better fit of the brace over the long term as compared to traditional casting. Also, there is no need to apply an initial plaster or fiberglass splint after reduction and later change to a cast, risking loss of reduction. With the PFFRB, one device may be used from beginning to end. For the surgeon, with the application of a splint or cast, molding of the cast typically takes 10-15 minutes. With the PFFRB, this process takes 15 seconds. The surgeon never has to take a splint or cast off and start over because the reduction is not as good as he or she would like. The surgeon simply has to make minor brace adjustments, taking mere seconds to do. There is never a risk of losing a reduction when transitioning from a splint to a cast. Fine adjustments can be made to obtain the perfect reduction, rather than settling for the “adequate” reduction.

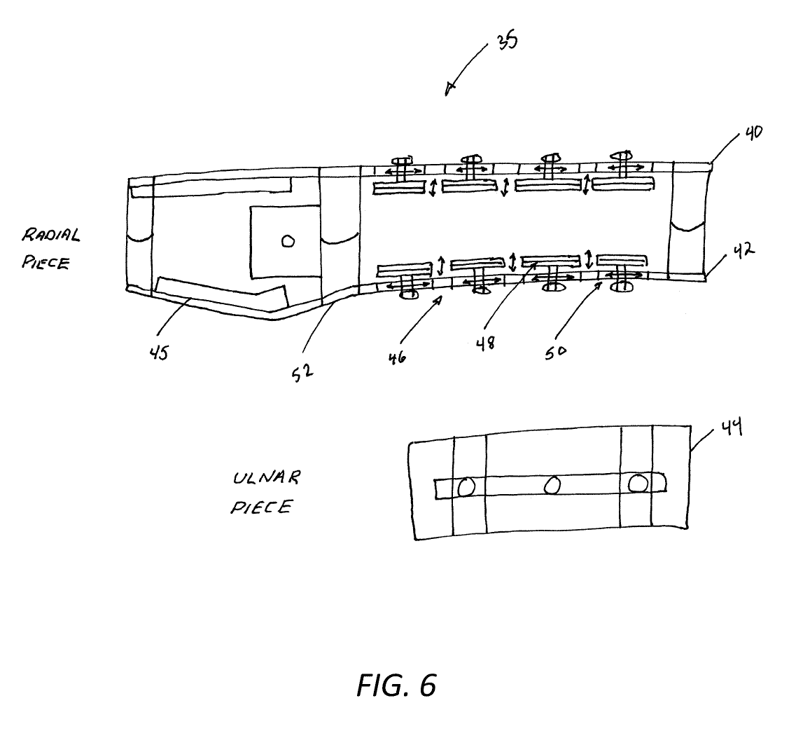

Referring now specifically to FIG. 6, in one exemplary embodiment, the PFFRB 35 of the present invention includes a plurality of splint members 40, 42, and 44 (including a rigid radial piece 40 and 44 and a rigid/flexible ulnar piece 46) that are shaped and sized to accommodate a forearm/wrist (distal radius/ulna, midshaft radius/ulna, etc.). Preferably, each of the splint members 40, 42, and 44 are made from a durable material that can get wet, and one or more of the splint members 40, 42, and 44 may include one or more compliant pads 45 that contact the forearm/wrist/hand for comfort. One or more reduction members 46 are deployable from the interior surface of one or more of the splint members 40, 42, and 44, such that the one or more reduction members 46 apply selective pressure on and provide positioning of one or more portions of the forearm. Both coarse and fine reduction members 46 may be provided, as appropriate and convenient, and reduction can be provided from multiple orthogonal directions about the forearm. This adjustably manipulates and reduces the forearm consistently while the brace 35 is in place, as verified by one or more x-rays or the like. Again, each of the one or more reduction members 46 may include one or more compliant pads 48 that contact the forearm/wrist/hand for comfort. Optionally, multiple reduction members 46 may utilize a common compliant pad 48. The reduction members 46 may be translated along the associated splint member 40, 42, or 44 via movement within a slot 50 manufactured into the splint member 40, 42, or 44, for example. The reduction members 46 may be deployed by rotational actuation of a screw or jack mechanism, for example, and prevented from translating via a suitable locking mechanism or the like. One or more localized air pockets may also be utilized, provided that an associated reduction member 46 is thereby deployed and positioned. Finally, the one or more splint members 40, 42, and 44 may be coupled together via one or more rigid structural members or compliant strap members 52, any of which may be adjustable. In this respect, the one or more splint members 40, 42, and/or 44 may be formed together, defining one or more voids.

The incidence of distal radius fractures (not counting midshaft forearm fractures that could also be treated by the PFFRB 35) is as follows:
10-14 year old boys—1.3% incidence
10-14 year old girls—0.8% incidence
5-9 year old boys—0.6% incidence
5-9 year old girls—0.82% incidence
15-19 year old boys—0.38% incidence
15-19 year old girls—0.21% incidence
   The estimated incidence among 5-17 year old boys and girls is thus 0.7%. There were 54 million children ages 5-17 in the US in 2010. Thus, an incidence of 0.7%×54 million=378,000 distal radius fractures annually in the US in patients ages 5-17.

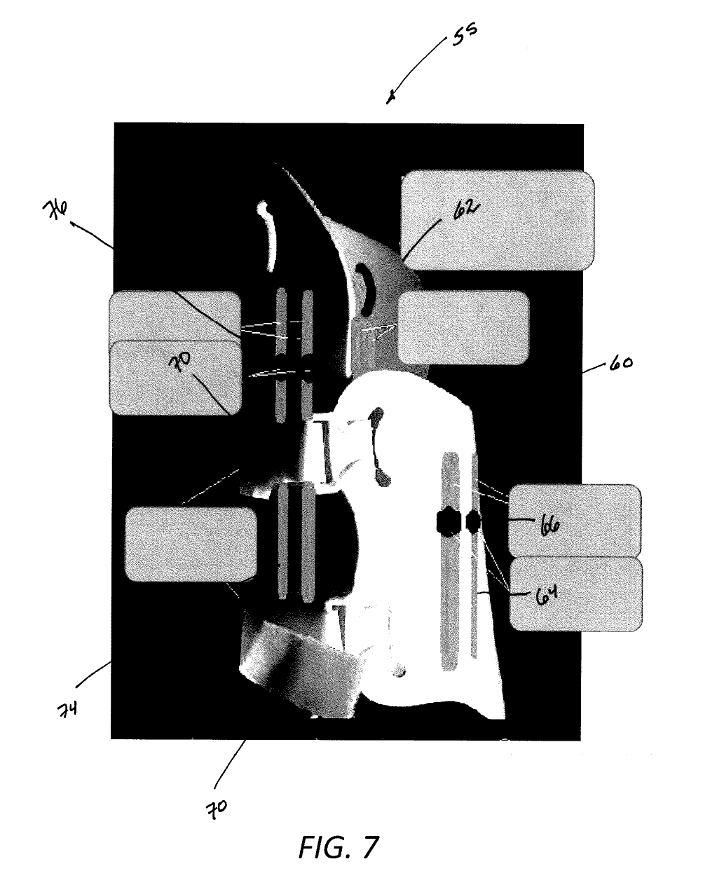

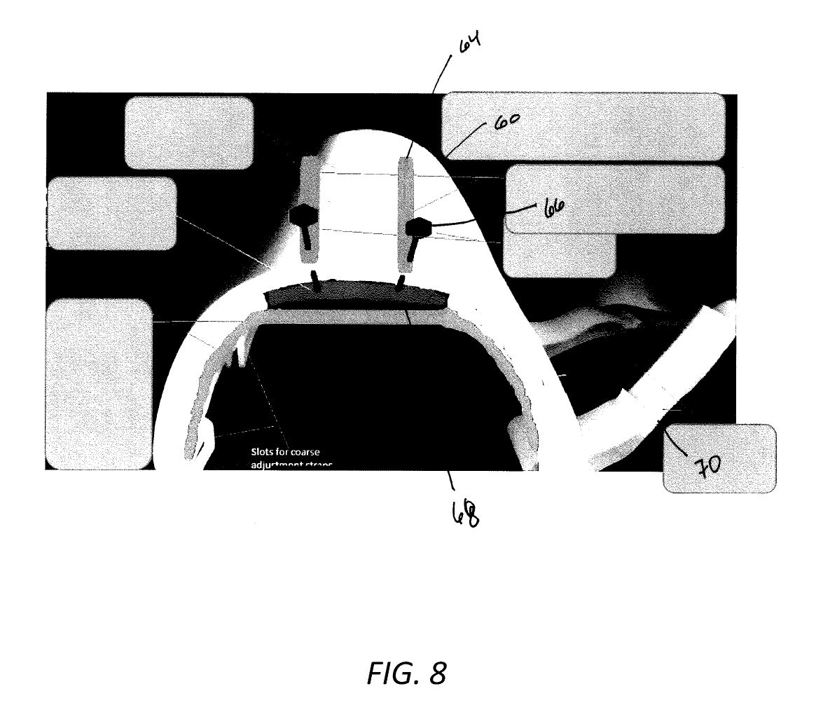

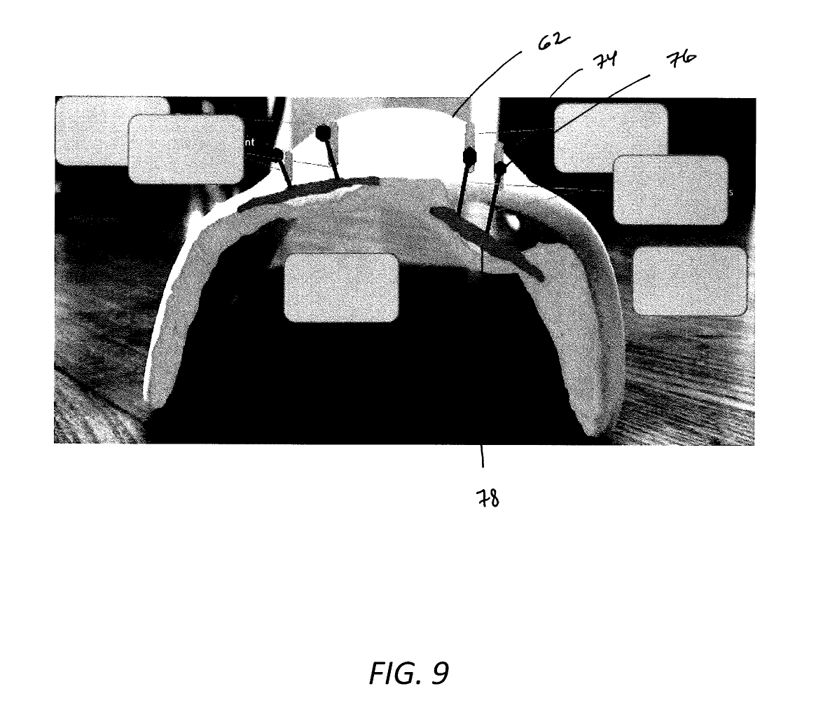

Referring now specifically to FIGS. 7-9, in one exemplary embodiment, the HFRB 55 of the present invention includes an inner (medial arm) piece 60 and a lateral piece 62 that are collectively designed to provide fragment specific reduction of humeral shaft fractures, simultaneously reducing and immobilizing humeral shaft fractures in a fracture specific way. It provides fragment specific reduction, unlike the traditional Sarmiento coaptation brace that is used today. The current Sarmiento brace only provides coarse reduction by circumferentially squeezing the soft tissue envelope around the humerus. This works fairly well for arms with thin soft tissue envelopes, achieving “adequate reduction.” However, adequate reduction is not perfect reduction. The upper arm will accommodate 10-20 degrees of anterior angulation and 10-30 degrees of varus. This amount of angulation may be considered acceptable for patients with low to moderate functional demands. The rate of union (for humeral shaft fractures) is generally high with nonoperative management, but the incidence of mild malunion is high. In a thin person, this degree of deformity may be visible. This malunion is prevented with the fragment specific reduction offered by the HFRB 55. In arms with larger soft tissue envelopes, the coarse adjustment provided by the Sarmiento brace is frequently not enough to provide acceptable reduction, leading to these requiring operative management. A similar situation is present with women with large breasts. The force of the large breast pushes the humeral shaft fracture into varus, again often leading to the need for operative management. The fragment specific reduction of the humeral fracture reduction brace would also work to counter this problematic external varus force.

HFRB reduction of humerus fractures provides the precise adjustment of fracture reduction pads and allows for more precise application of reduction pressure as compared to the global compression of a Sarmiento style brace, thus often improving the initial reduction. Easy fine tuning of the pressure applied by fracture reduction pads allows for easy correction when the fracture is not quite perfectly reduced. A surgeon can literally dial in the reduction under fluoroscopic guidance.

The inner piece 60 includes a plurality of grooves 64 for the positioning and deployment of a plurality of fine adjustment screws 66, such that one or more reduction pads 68 may be deployed. Coarse adjustment may be provided by straps or the like 70. Similarly, the lateral piece 62 includes a plurality of grooves 74 for the positioning and deployment of a plurality of fine adjustment screws 76, such that one or more reduction pads 78 may be deployed. Coarse adjustment may again be provided by straps or the like 70.

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims.

What is claimed is:

1. A fracture reduction brace, comprising:
one or more rigid splint members configured to be disposed adjacent to or about an anatomical structure; and
one or more reduction members deployable from an interior surface of the one or more rigid splint members into the anatomical structure such that the one or more reduction members apply pressure to the anatomical structure thereby reducing an associated fracture.

2. The fracture reduction brace of claim 1, wherein the one or more rigid splint members comprise a plurality of rigid splint members.

3. The fracture reduction brace of claim 2, wherein the plurality of rigid split members are coupled together via one or more straps or other coarse adjustment mechanisms.

4. The fracture reduction brace of claim 2, wherein the plurality of rigid split members are coupled together via one or more structural members.

5. The fracture reduction brace of claim 1, wherein each of the one or more reduction members is deployable from the interior surface of an associated rigid splint member via an actuation mechanism that protrudes through the associated rigid splint member.

6. The fracture reduction brace of claim 5, wherein the actuation mechanism comprises a screw assembly that is actuated via rotational motion.

7. The fracture reduction brace of claim 1, wherein each of the one or more reduction members is translatable along the interior surface of an associated rigid splint member.

8. The fracture reduction brace of claim 7, wherein each of the one or more reduction members is translatable along the interior surface of the associated rigid splint member via a groove manufactured into the associated rigid splint member.

9. The fracture reduction brace of claim 1, further comprising one or more compliant pads coupled to the interior surface of the one or more rigid splint members.

10. The fracture reduction brace of claim 1, further comprising one or more compliant pads coupled to the one or more reduction members.

11. A method for providing and using a fracture reduction brace, comprising:
providing one or more rigid splint members configured to be disposed adjacent to or about an anatomical structure;
providing one or more reduction members deployable from an interior surface of the one or more rigid splint members into the anatomical structure such that the one or more reduction members apply pressure to the anatomical structure thereby reducing an associated fracture;
disposing the one or more rigid split members securely adjacent to or about the anatomical structure; and
deploying the one or more reduction members from the interior surface of the one or more rigid splint members into the anatomical structure.

12. The method for providing and using the fracture reduction brace of claim 11, wherein the one or more rigid splint members comprise a plurality of rigid splint members.

13. The method for providing and using the fracture reduction brace of claim 12, wherein the plurality of rigid split members are coupled together via one or more straps or other coarse adjustment mechanisms.

14. The method for providing and using the fracture reduction brace of claim 12, wherein the plurality of rigid split members are coupled together via one or more structural members.

15. The method for providing and using the fracture reduction brace of claim 11, wherein each of the one or more reduction members is deployable from the interior surface of an associated rigid splint member via an actuation mechanism that protrudes through the associated rigid splint member.

16. The method for providing and using the fracture reduction brace of claim 15, wherein the actuation mechanism comprises a screw assembly that is actuated via rotational motion.

17. The method for providing and using the fracture reduction brace of claim 11, wherein each of the one or more reduction members is translatable along the interior surface of an associated rigid splint member.

18. The method for providing and using the fracture reduction brace of claim 17, wherein each of the one or more reduction members is translatable along the interior surface of the associated rigid splint member via a groove manufactured into the associated rigid splint member.

19. The method for providing and using the fracture reduction brace of claim 11, further comprising providing one or more compliant pads coupled to the interior surface of the one or more rigid splint members.

20. The method for providing and using the fracture reduction brace of claim 11, further comprising providing one or more compliant pads coupled to the one or more reduction members.
